(19) United States
(12) Patent Application Publication
(10) Pub. No.: US 2006/0184070 A1
(43) Pub. Date: Aug. 17, 2006

(54) External ultrasonic therapy

(76) Inventors: Douglas R. Hansmann, Bainbridge Island, WA (US); Peter Rule, Los Altos, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/272,022

(22) Filed: Nov. 11, 2005

Related U.S. Application Data
(60) Provisional application No. 60/627,469, filed on Nov. 12, 2004.

(51) Int. Cl. A61H 1/00; A61B 8/12

(52) U.S. Cl. 601/2; 600/439

(57) ABSTRACT

A coupling pad can be used with a transducer element. Ultrasound produced by the transducer element can be propagated through the coupling pad to a patient. In one arrangement, the pad is a pouch containing a pliant substance. In other arrangements, the pad is a somewhat solid member formed of a material that transmits ultrasound waves efficiently. The pad may be configured to fit between a patient's skin and a transducer element so that output from the transducer element passes through the pad to a treatment site of the patient.

### RELATED APPLICATIONS

This application claims the priority benefit under 35 U.S.C. § 119(e) of U.S. Provisional Application No. 60/627,469, filed Nov. 12, 2004, which is hereby incorporated by reference in its entirety.

### BACKGROUND OF THE INVENTION

1. Field of the Invention

The invention relates to ultrasonic therapy, and in particular, to therapy that utilizes an external ultrasonic device configured to deliver therapeutic ultrasonic energy to a patient.

2. Description of the Related Art

Ultrasonic energy may be applied to selected regions within a body to provide a therapeutic effect. In some applications, the ultrasound is generated from an extracorporeal ultrasonic transducer. Examples of such applications include ultrasonic ablation of tissue as shown in U.S. Pat. No. 4,858,613, “Localization and Therapy System for treatment of spatially oriented focal disease”, issued Aug. 22, 1989, to Fry et al.; fracture of kidney stones, as shown in U.S. Pat. No. 4,539,989, “Injury Free Coupling of Therapeutic Shock Waves”, issued Sep. 10, 1985 to Forssmann et al; heat therapy, as shown in, e.g., U.S. Pat. No. 4,586,512, “Device for Localized Heating of Biological Tissue, issued May 6, 1986 to Do-huu; and destruction of thrombi, as shown, e.g., U.S. Pat. No. 5,509,896 “Enhancement of Sonothrombolysis with External Ultrasound, issued Apr. 23, 1996 to Carter et al. The mechanisms of action for these applications require acoustic intensity levels sufficient to cause significant heating or mechanical disruption or destruction of tissue preferably only within a localized region.

The acoustic intensities used for treatment in the localized region of the body range from 0.5 to 100′s of watts per cm² at the internal treatment site, at frequencies in the 100 kHz-2 MHz range. Prolonged exposure to intense acoustic fields causes tissue destruction through heating or mechanical action. Thus, it may be important that the acoustic field be controlled so that only the target tissue receives prolonged exposure. Ultrasonic energy may have to pass through intervening layers of energy-absorbing tissue, like the skull, in order to reach the area targeted for treatment. This often causes heating of those intervening layers. For example, bone absorbs ultrasound at least thirty times more readily than brain tissue. Thus, to avoid undue skull heating, acoustic intensities at the skull are typically kept relatively low.

### SUMMARY OF THE INVENTION

In some embodiments, a coupling pad can be used with a transducer element. Ultrasound produced by the transducer element can be propagated through the coupling pad to a patient. In one arrangement, the pad is a pouch containing a pliant substance. The pad may be configured to fit between a patient's skin and a transducer element so that output from the transducer element passes through the pad to a treatment site of the patient. In some variations, the treatment site is tissue in the patient's head.

In some embodiments, a pad is used with a therapeutic ultrasonic treatment device configured to apply ultrasonic energy to a patient's head. The pad comprises a pouch defining a chamber. An ultrasonic coupling media is disposed within the chamber of the pouch. The pad is configured to fit between the patient's skin and a transducer element of the treatment device.

The pad can be somewhat pliant. In some embodiments, the pad is adapted to conform to the topology of the patient's skin. The coupling media disposed within the chamber of the pouch can comprise a gel. The pad is configured to provide a gap between the transducer element and the patient's skin. In some embodiments, the pad is a disposable pad suitable for at least one ultrasound treatment. In other embodiments, the pad is a multi-use pad.

In yet other embodiments, a system for outputting an ultrasound field to a person's head is provided. The system comprises a transducer element configured to apply an ultrasound field to a treatment site in a person's head. A pad includes a covering containing an ultrasonic coupling agent. The pad is sized and configured to separate the transducer element and the skin of the patient's head. In some variations, the coupling agent comprises an ultrasound gel.

The system can further comprise a second transducer element and a second pad. The second pad includes a covering that contains a coupling agent. The second pad is sized and configured to separate the second transducer element and the skin of the patient's head.

In some embodiments, a headset is configured to hold the transducer elements about the person's head such that the pads are compressed between corresponding transducer elements. The pads can be coupled to the headset. In other embodiments, the pads and headset are separatable.

In some embodiments, a method of delivering ultrasound to a patient is provided. The method comprises positioning an ultrasound transducer element relative to a treatment site of a patient. A pliant pad is positioned between the transducer element and the patient. A clot removing agent is delivered to the treatment site. Ultrasound is delivered from the transducer element through the pad and to the treatment site. In some variations, a plurality of ultrasound transducer elements is positioned relative to the treatment site of the patient. Pliant pads are positioned between the transducer elements and the patient. In some variations, pliant pads are compressed between the transducer elements and the patient's skin. In some variations, the pads comprise a bag containing gel.

### BRIEF DESCRIPTION OF THE DRAWINGS

FIG. 1 illustrates a cross section of skull, and a geometrically focused transducer to achieve high intensities somewhere within it.

FIG. 2 depicts a plan view of a cranial vault, showing an interference pattern that is generated by acoustic waves being reflected back and forth within the cranial vault.

FIG. 3A depicts a block diagram of an apparatus that can deliver acoustic waves.

FIG. 3B illustrates one type of modulation that results in aperture shifting.

FIG. 4A illustrates a headset connected to system electronics via a cable. The headset is positioned on a patient's head.

FIG. 4B is a front view of a patient wearing a headset that can deliver ultrasonic energy.

FIG. 4C is an enlarged front view of a pad and a transducer element of the headset of FIG. 4B.

FIG. 4D is a cross-sectional view of the pad of FIG. 4C.

FIG. 4E is a frontal view of one embodiment of a pad for use with the headset of FIG. 4B.

FIG. 4F is a front view of an another embodiment of a pad for use with the headset of FIG. 4B.

FIG. 5 is a front view of a patient wearing a headset adapted to deliver ultrasound and a pad surrounding a portion of the patient's head. The pad is positioned between the headset and the patient.

FIG. 6 illustrates an ultrasonic apparatus applied to a leg of a patient.

### DETAILED DESCRIPTION OF THE PREFERRED EMBODIMENT

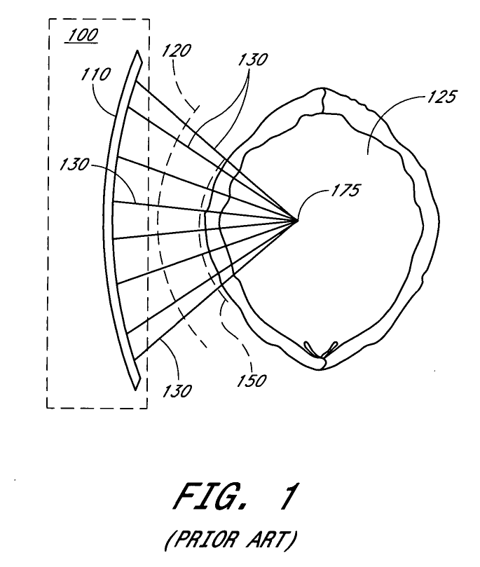

In one particular embodiment described below, an external device can deliver ultrasound, which enhances the effect of a drug configured to treat an occlusion of a blood vessel within the brain. Such an embodiment is particularly useful for treating victims of ischemic stroke. The external ultrasonic device is preferably configured to produce an acoustic field about the treatment site. For stroke treatment, the acoustic field delivered to the skull can be shaped by geometric focusing, using either physical or electronic lenses to reduce or avoid undue skull heating. FIG. 1 illustrates one embodiment of this type of external ultrasound device. As shown, the device includes an ultrasound transducer 100 that contains a lens structure 110 that produces a concave shaped wave front 120 that converges along paths 130 to a point of intended focus 175 after transiting the skull 125. The acoustic intensity at the convergence point 175 is many times higher than it acoustic intensity at the penetration area 150 of skull 125. See also U.S. Pat. No. 6,575,922 which is hereby incorporated by reference herein.

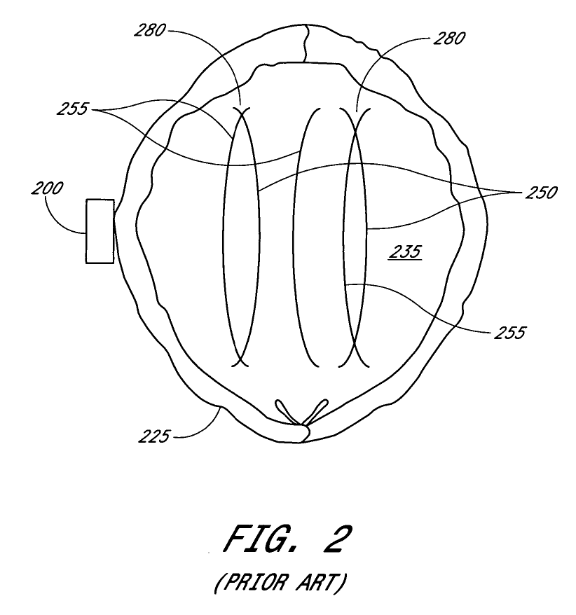

Another embodiment of a external ultrasonic device utilizes the observation that many body structures (e.g., the skull) act as resonators. For example, FIG. 2 illustrates an acoustic field in a skull. At frequencies in the 0-500 kHz range, there is little attenuation of longitudinal acoustic waves in the brain or skull, and if reflection at a boundary layer is near total, then acoustic waves pass back and forth through tissue many times creating a trapped mode resonator.

The ultrasound transducer 200 emits wave fronts 250 that transit the skull 225. Due to low acoustic loss in the tissue 235 in the cranial vault, the waves travel to the other side of the skull, where they are reflected as wave 255 due to the differing acoustic impedance of the bone and air which forms the skull and its outside boundary. These reflected waves 255 again travel across the cranial vault and again are reflected by the bone and air interfaces, and return across the cranial vault. This process is repeated many times, and builds up to the point where the internal acoustic energy losses in the cranial vault and the reflections at the skull balance the acoustic energy applied by transducer 200. At points 280 where the acoustic waves intersect, pressure nodes and anti-nodes are formed, depending on whether the wave fronts interfere out of phase or in phase, respectively. A common measure of the resonant property of a system is its quality factor, Q, defined as 2π times the ratio of stored energy to the lost energy per cycle. In practice, Q′s from 10 to more than 100 can be obtained with node to anti-node pressure ratios of from 10 to more than 100.

A body part can be treated with acoustic waves below 500 kHz, and preferably below 100 kHz, as a trapped mode resonator. Such a resonator can exhibit a high Q (e.g., a Q of 10 or more) at certain frequencies that cause wave front interference from multiple reflections to add up in phase. Examples of trapped mode resonators within a body include: (a) the cranial vault bounded by air, bone, and neck tissue; (b) arms; (c) legs; and (d) the thorax, all of which are bounded by air and other tissues. In high Q resonators, very high pressures can be achieved in the resonator cavity for a very modest input power U. The differing impedance of skull and air from brain assures that there will be internal reflections, thereby causing the cranial vault to act as a resonator. At frequencies below 500 kHz, little acoustic power need be delivered to the skull to maintain the acoustic field in the brain, because there is little skull or brain heating caused by absorption or other losses. The losses can be reduced or substantially eliminated by using a coupling pad disclosed in detail below.

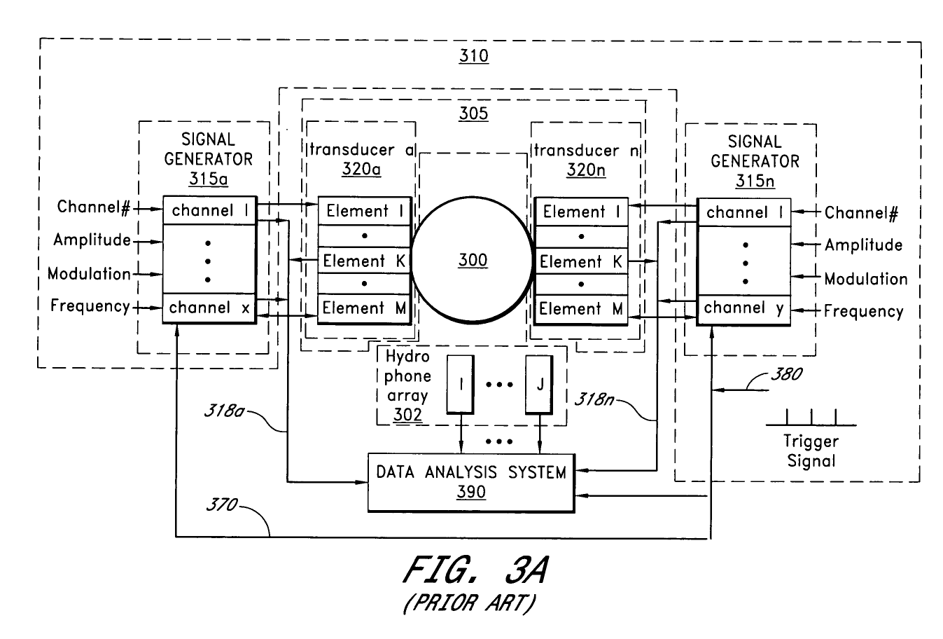

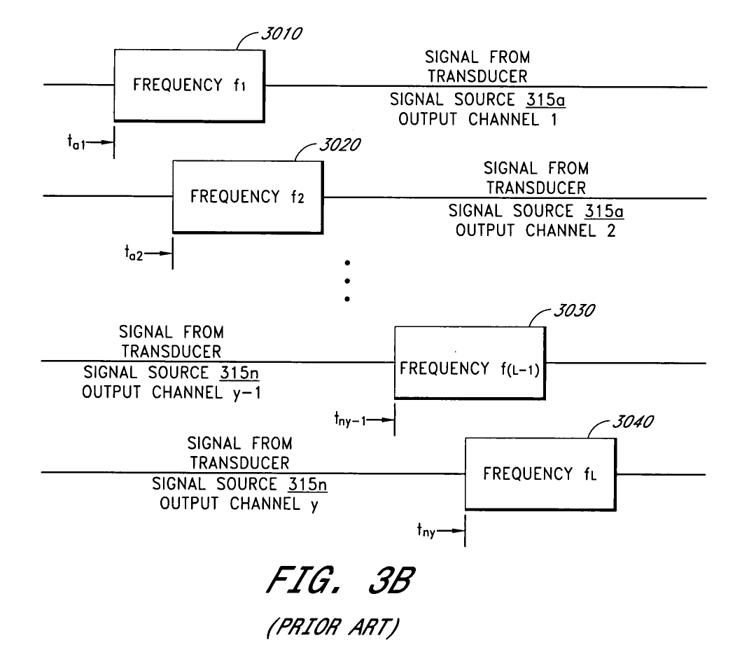

FIG. 3A is a block diagram of an exemplary apparatus that operates to generate acoustic waves. Acoustic generator 305 consists of transducers 320a . . . n that are placed in contact with the body part 300 that is to be excited as a resonator. Each transducer consists of one or more transducer elements 320al . . . M, and 320nl . . . L as shown on the diagram. Transducer element 320al, for example, may be used exclusively as a transmitter, i.e., it is used only to transmit acoustic energy into the body. Transducer element 320ak, for example, may be used as a hydrophone, i.e. it may be used only to receive acoustic energy. Transducer elements 320aM and 320nL may be used bidirectionally, i.e., they both transmit and receive acoustic energy.

Acoustic generator 305 is connected to a generator signal source 310. The generator signal source 310 is composed of one or more signal sources 315a . . . x, up to one for each transducer 320a . . . n. Each transducer signal source may have one or more output channels. For example, the signal generator 315a has channel 1 . . . channel x. These channels are connected to transmitter transducer elements or to bi-directional transducer elements or to both, and are also connected to a signal bus, 318a . . . n. Transducer elements k, which are used as hydrophones, are not typically connected to a signal generator, but are connected to a signal bus 318a . . . n. Measurements of impedance and also power for any element may be made by data analysis system 390 which is also connected to these signal busses.

For each output channel of a transducer element's signal source 315a . . . 315n, the drive signal amplitude, modulation characteristics, signal waveform type, and/or frequency may be independently specified. Trigger signal 380, shown as a train of synchronizing pulses, but which may be some other synchronizing signal, is generated within signal source 310 and synchronizes the signal generators 315a . . . n and also the data analysis system 390, via the buss 370. The system also includes an array of hydrophones 302, consisting of k independent hydrophones which are placed in contact with the body part that is to driven as a resonator. These hydrophones are also connected to the data analysis system 390, so that their outputs can be analyzed.

Placement and movement of the nodes and anti-nodes within the resonator are controlled by controlling the amplitude, frequency or phase, or any combination thereof for one or more transducer signal generator output channels. It is specifically noted that amplitude modulation may be used to electronically move one or more active apertures, which also moves the locations of the nodes and anti-nodes within the resonator. For example, referring to FIG. 3B, signal bursts 3010, 3020, 3030, 3040 are shown. These are tone bursts which are generated by amplitude modulating each carrier signal f1 . . . fn1 by the appropriate envelope, e.g., a rectangle function, delayed by that appropriate delays t1 . . . tn1. By making the delays unequal, different elements are turned on at different times. Since the active aperture at any time is composed of only those elements that are being excited, the active aperture is moved electronically, thereby moving the positions of the nodes and anti-nodes within the resonator.

As discussed above, the signal generators 315 provide electrical driving signals for the acoustic transducer drivers 320. These signals may comprise, e.g., sinusoidal waves of defined amplitude, frequency, phase or waveshape, or may comprise pulses of defined amplitude and duration. The signal generators may comprise self-contained units in which the control variables (amplitude, frequency, phase, pulse height, pulse duration, waveshape, etc.) are set by the user by manipulating control knobs that set the control variables. Alternatively, signal generators may be responsive to a control program, stored either internally within the system or externally to it, which defines and controls the desired parameters. Signal generators of both types are known and, indeed, are commonly available as commodity items.

As illustrated in FIG. 3A, the driving signals of signal generators 315 may be controlled from an external programmed controller and data analysis system 390. Preferably the system 390 comprises a programmable data processor, e.g., a “Personal Computer” into which the user may enter a control program specifically prepared by him/her to control the variables of interest for the particular treatment or experiment. Preferably, the control program enables the user to vary the driving parameters in real time, if desired, to accommodate specific patient or experimental conditions. To facilitate positioning of the nodes and anti-nodes, a joystick is advantageously coupled to the controller for rapid change of one or more of the control parameters, and a video monitor is provided on which the region of interest is displayed showing either the actual location of an acoustic maxima or a computed location in accordance with the specific control parameters characterizing the signal generator output as any given moment. The system 390 may also serve to analyze data generated during the treatment or experiment.

The arrangement of FIGS. 3A and 3B provides increased flexibility in positioning and controlling the energy maxima and minima with a desired region of a skull or other body cavity. Advantageously, the energy can be precisely controlled to treat local regions of the head effected by, e.g., a stroke.

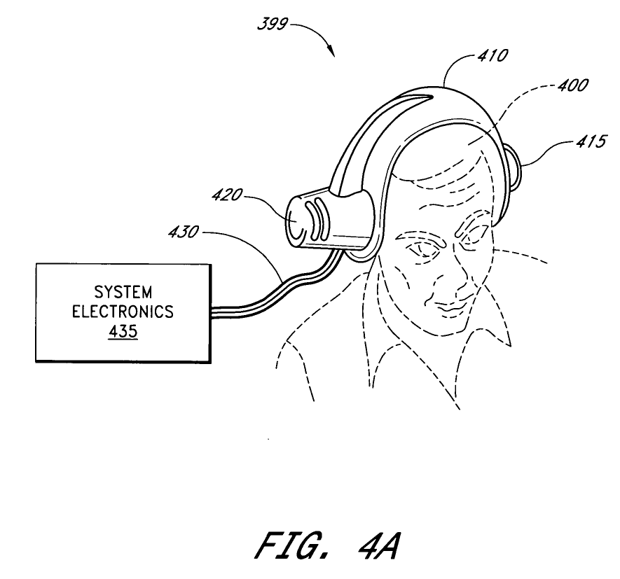

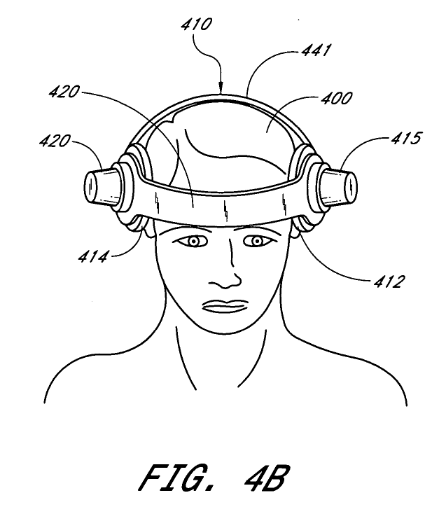

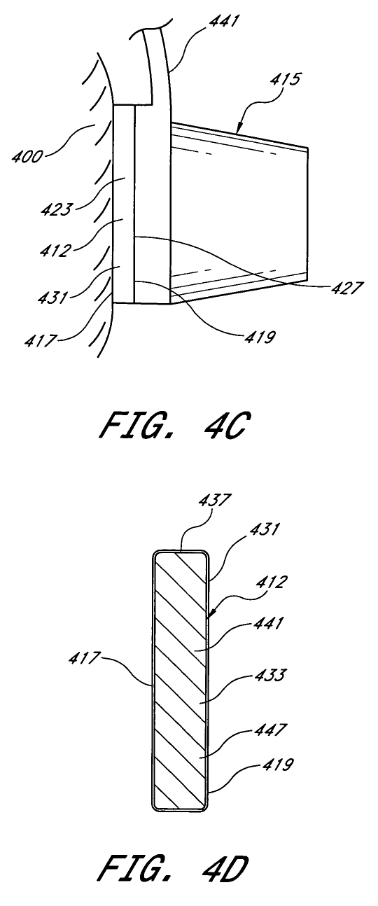

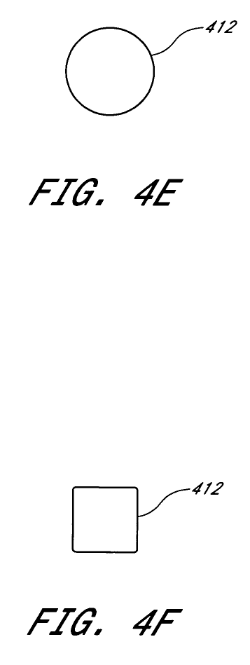

FIG. 4A illustrates an embodiment of system 399 comprising a transducer holder 410, which in the illustrated embodiment is in the form of a headset. As will be described below, the headset 410 is configured to be worn by a patient. The headset 410 supports transducers that deliver one or more acoustical fields to the head of the patient. The system 399 also comprises one or more coupling pads. The coupling pads can be configured to facilitate the transmission of ultrasound waves emitted by the headset 410 to the patient.

As described above, the headset 410 is mounted on the head 400 of a subject and is used to produce one or more acoustic fields inside a human skull, with the cranial vault preferably being the resonant cavity. In the illustrated embodiment, the headset 410 comprises transducer elements 415, 420. A cable 430 leads to the system electronics 435 and is connected to the headset 410. The cable 430 provides communication between the transducers of the headset 410 and the electronics 435.

Transducers 415 and 420 are mounted in the headset 410 such that they press on opposite sides of the head 400 above the ears. The illustrated transducers 415 and 420 are generally diametrically spaced about the patient's head 400. However, the transducers 415 and 420 can be spaced at any suitable location about the patient based on the desired treatment. The headset 410 can have any number of transducers for producing the desired ultrasound field. For example, the headset 410 may comprise three transducers that are spaced evenly or unevenly about the patient's head. In alternative embodiments, a single transducer is mounted to a headset. Thus, any number of transducers can be employed depending on the treatment.

As shown in FIG. 4B, a coupling member or pad 412 is disposed between at least one of the transducers of the headset 410 and the patient's head 400. In one embodiment, a plurality of coupling pads are disposed between the headset 410 and the patient. Preferably, at least one pad is interposed between each of the transducers of the headset 410 and the patient's head 400. As explained below, the pads may advantageously provide desirable acoustic coupling between a corresponding transducer and the patient. Thus, acoustic waves can be transmitted through the pads to the treatment site, preferably without substantial reduction in the intensity of the acoustic waves.

With continued reference to FIG. 4B, the headset 410 can have a strap or front portion 420 that securely fastens the headset 410 to the patient. In the illustrated arrangement, pads 412, 414 and corresponding transducers 415, 420 are disposed over at least a portion of the patient's ears. However, the headset 410 can be oriented and positioned on the head 400 so that the pads and transducers are located at any other positions along the patient's head 400.

FIG. 4C is a front view of the pad 412 and the transducer 415 of the headset 410. The pad 412 is preferably plaint and sized so as to fit between the headset 410 and the patient. The pad 412 is sandwiched between the transducer 415 and the patient, and has an inner face 417, an outer face 419, and a side wall 423 therebetween. When the headset 410 is worn by a patient, at least a portion of the inner face 417 engages the patient's skin. In some embodiments, for example, substantially the entire inner face 417 contacts the skin of a patient. The headset 410 can be biased inwardly towards the patient. Such headsets can continuously press the coupling pad 412 against the patient's skin to ensure proper acoustical coupling is maintained during treatment.

The pad 412 can be permanently or temporarily coupled to the headset 410. For example, the outer face 419 of the pad 412 can be permanently or temporarily coupled to the transducer 415 by fasteners, adhesives, clips, snaps, hook-and-loop fasteners (e.g., Velcro), combinations thereof, or the like. In other embodiments, the pad 412 may be held between the transducer 415 and the patient only by compressive forces created by the headset 410.

The pad 412 can be coupled to the headset 410 for one or more treatment cycles and/or for treating one or more patients. In some embodiments, the pad 412 is configured for a single use by a single patient. In such embodiments, the disposable, one-time use pad 412 may be provided in a sterile free package. The pad 412 can be removed from the sterile package and attached to the transducer, or otherwise secured between the patient and transducer. The transducer can apply ultrasonic energy to the patient via the pad 412. After use, the pad 412 may be detached from the transducer 415 and then discarded. In alternative embodiments, the pad 412 is a multiuse pad that can be used any number of times as desired.

As mentioned above, the pad 412 is preferably plaint so that the pad 412 conforms to the topology of the patient's skin to provide a more efficient acoustical coupling between the transducer and the treatment area. The topology of the patient's skin, in general, will be different from the topology of the ultrasound transducer, such as the transducer surface 427. However, the pad 412 can be compressed between the transducer 415 and the head 400, thereby conforming the pad 412 to the topology of the patient skin and facilitating efficient transmission of ultrasound waves from the transducer 415 through the pad 412 and to the target treatment tissue.

As illustrated in FIG. 4D, the pad 412 comprises a bag or pouch 431 filled with a viscous or flowable substance, such as fluid or gel 433. However, the substance can be any suitable substance for propagating output from the headset 410 to the patient. For example, the substance 433 can be a pliant rubber, plastic, polymer, elastomer, and/or the like. Preferably, the pad 412 is configured to maintain a gap between the transducer 415 and the patient, even when the headset comprises the pad 412.

In the embodiment of FIG. 4D, the pouch 431 includes walls 437 that define a chamber 447 is configured to contain the gel 431 and allows the pad 412 to deform to the contours of the patient's head 400. The wall 437 of the pouch 431 may comprise polymers (e.g., polyethylene, polypropylene, and/or the like) and/or any other material suitable for containing a substance for propagating ultrasound waves. The pad 412 can comprise a gel having similar or different characteristics (e.g., viscosity, density, or the like) as typical ultrasound gel. The fluid or gel within the pouch 431 can be any substance suitable for transmitting ultrasound. The size and configuration of the pouch 431 and the type of substance within the pouch 431 can be chosen to achieve the desired acoustical properties of the pad 412. In alternative embodiments, the pad 412 comprises a single material. For example, the pad 412 can be a solid body comprising a polymer, rubber, or other material.

The pad 412 can have any suitable shape. FIGS. 4E and 4F are elevation side views of pads 412. The pad 412 of FIG. 4E has a generally circular shape. The pad 412 of FIG. 4F has a generally polygonal shape. Other configurations of pads can also be used.

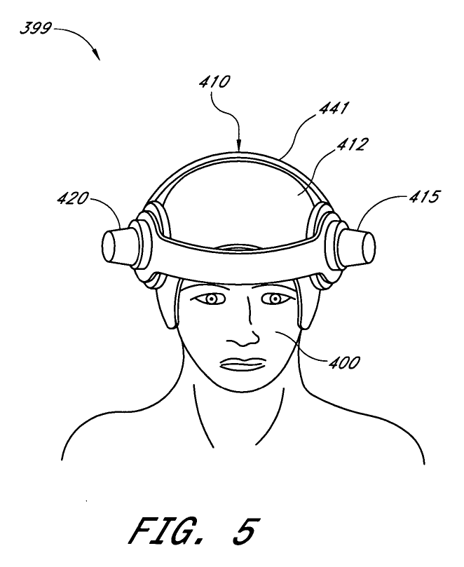

FIG. 5 illustrates another embodiment of a pad used with the headset 410. The illustrated pad 412 is sized and configured to fit onto and surround a portion of a patient's head. For example, the pad 412 can fit over the ears and upper portion of the patient's head. This helmet pad 412 ensures that the ultrasound waves are not delivered directly to the patient. The pad 412 is preferably interposed between at least one of the transducer elements (e.g., transducers 415, 420) of the headset 410 and the patient. In the illustrated embodiment, the pad 412 separates a plurality of transducers 415, 420 from the patient. Advantageously, the helmet pad 412 can be used for multiple treatment cycles wherein transducers are positioned at different locations along the patient's head 400. Thus, the pad 412 can remain in generally the same position while transducers are located in multiple positions. Of course, the helmet pad 412 can be a single-use or multi-use pad.

Optionally, the pad 412 may have indicia for the physician. The indicia can be located on the surface of pad 412 and can indicate desired positioning of the headset 410 relative to the pad 412. The indicia can indicate any information that a physician may deem useful. The indicia can be printed, adhered, and/or embossed on the pad 412.

With reference to FIG. 4B and FIG. 5, the bias provided by a head band 441 of the headset 410 causes the transducers 415 to compress the corresponding pad or pads.

Although not shown, it is contemplated that the outer surface of the pad may also engage a layer of acoustic coupling media (e.g., a coupling agent or gel) to ensure good acoustic coupling between a transducer and the treatment site. Additionally, water, saline, water-based solutions, ultrasound gels or any other suitable coupling media can be used in combination with the pads disclosed herein. For example, a coupling media can be spread on the inner surface 417 of the pad 412 of FIGS. 4C and/or FIG. 5 to further enhance the propagation of ultrasound waves to the patient. The coupling media can be spread before and/or during the ultrasound treatment. It is contemplated that one or more layers of acoustic coupling gel can be disposed between the patient and the pad 412 and/or the pad 412 and the transducer 415.

The pad 412 can be similar or different than the second pad 414. Thus, the relationship between the second pad 414 and the second transducer 420 may be similar or different than the relationship between the first pad 412 and the first transducer 415.

The headset 410 can be used to aid in the delivery of drugs that are typically used to treat complications due to stroke, Parkinson's disease, or other brain disorders or diseases. For example, the headset 410 can be used to promote drug and/or gene preparations to pass through blood brain barriers. The headset 410 also can be used to treat brain tumors (e.g., primary and/or matastatic tumors). In particular, the ultrasound energy can be used as part of a clot dissolution treatment. In one embodiment, the patient is treated with a clot removing drug. The drug can be administered intravenously or through a drug delivery catheter positioned near or within the brain. The ultrasonic energy is the applied to the brain using an ultrasonic device such as the ultrasonic devices described herein. The ultrasonic energy enhances the therapeutic effect of the clot removing drug. In other embodiments, the ultrasonic energy may be used without the clot removing drug. Examples of clot removing drugs include but are not limited to thrombolytic agents (such as, for example, Heparin, Uronkinase, Streptokinase, Tissue Plaminogen Activator (TPA) and BB-10153, which is manufactured by British Biotech), anti-thrombis drugs, and/or other drugs and enzymes.

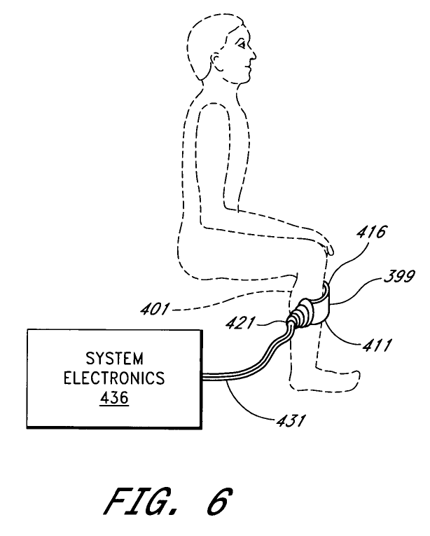

FIG. 6 depicts a system 399 applied to a body appendage, in this case a lower leg which is the resonant structure. The system 399 includes a transducer holder 411 that is mounted on the lower leg 401 of a subject. Receiving transducer 416 and exciting transducer 421 are mounted in transducer holder 411 and are pressed against opposite sides of the leg. Cable 431 extends between the system399 and the system electronics 436. Pads can be interposed between corresponding transducers 416, 421 and the contact area on the patient. The pads can be similar or different than the pads 412 and/or 414. In one embodiment of use, the system 399 is used to treat thrombus or other blockages in the blood vessels in the leg or other appendages.

All of the patents mentioned herein are incorporated by reference in their entire and made a part of this specification. Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein.

The method which is described and illustrated herein is not limited to the exact sequence of acts described, nor is it necessarily limited to the practice of all of the acts set forth. Other sequences of events or acts, or less than all of the events, or simultaneous occurrence of the events, may be utilized in practicing the embodiments disclosed herein.

What is claimed is:

1. A pad for use with a therapeutic ultrasonic treatment device configured to apply ultrasonic energy to a patient's head, comprising:
a pouch defining a chamber; and
an ultrasonic coupling media disposed within the chamber of the pouch;
wherein the pad is configured to fit between a patient's skin and a transducer element of the treatment device.

2. The pad of claim 1, wherein the pad is pliant and conforms to the topology of the patient's skin.

3. The pad of claim 1, wherein the ultrasonic coupling media comprises gel.

4. The pad of claim 1, wherein the pad is configured to provide a gap between the transducer element and the patient's skin.

5. The pad of claim 1, the treatment device comprises a headset configured for ultrasound treatment of a patient's head.

6. The pad of claim 5, wherein the headset comprises a pair of transducers.

7. The pad of claim 1, wherein the pad is a disposable pad suitable for at least one ultrasound treatment.

8. A system for outputting an ultrasound field to a person's head, comprising:
a transducer element configured to produce an ultrasound field into a treatment site in a person's head; and
a pad including a covering containing an ultrasonic coupling agent, the pad being sized and configured to separate the transducer element and the skin of the patient's head.

9. The system of claim 8, further comprising a second transducer element and a second pad, the second pad including a covering containing a coupling agent, the second pad being sized and configured to separate the second transducer element and the skin of the patient's head.

10. The system of claim 9, further comprising a headset configured to hold the first and second transducer elements about the person's head such that the first and second pads are compressed between the corresponding transducer elements and the patient.

11. The system of claim 8, wherein the coupling agent comprises an ultrasound gel.

12. A method of delivering ultrasound to a patient's head, the method comprising:
positioning an ultrasound transducer element relative to a treatment site of a patient;
positioning a pliant pad between the transducer element and the patient, the pliant pad acoustically coupling the transducer to the patient;
delivering an clot removing agent to the treatment site; and
delivering ultrasound from the transducer element through the pad to the treatment site.

13. The method of claim 12, further comprising:
positioning a plurality of ultrasound transducer elements relative to the treatment site of a patient; and
positioning a corresponding pliant pad between each of the transducer elements and the patient.

14. The method of claim 12, further comprising compressing the pliant pad between the transducer element and the patient's skin.

15. The method of claim 12, wherein the pad comprises a bag containing gel.
